(19) United States
(12) Patent Application Publication
(10) Pub. No.: US 2009/0249863 A1
(43) Pub. Date: Oct. 8, 2009

(54) VAPOR PHASE DECOMPOSITION DEVICE FOR SEMICONDUCTOR WAFER POLLUTANT MEASUREMENT APPARATUS AND DOOR OPENING AND CLOSING DEVICE

(75) Inventors: Ho Jin Kim, Icheon-si (KR); Hyoung Bae Kim, Seongnam-si (KR)

Correspondence Address: LEXYOUME IP GROUP, LLC, 5180 PARKSTONE DRIVE, SUITE 175, CHANTILLY, VA, 20151, US

(73) Assignee: KOREA TECHNO CO., LTD, Gwangju-si (KR)

(21) Appl. No.: 12/206,107

(22) Filed: Sep. 8, 2008

(30) Foreign Application Priority Data
Apr. 3, 2008 (KR) 10-2008-0031128

(51) Int. Cl. G01N 33/00

(52) U.S. Cl. 73/31.07

(57) ABSTRACT

Provided is a vapor phase decomposition (VPD) device for a semiconductor wafer pollutant measurement apparatus and a door opening and closing device thereof. The VPD device includes: a rectangular vessel-shaped main body of the VPD device and a door which covers in a sealed form or opens the wafer inlet of the VPD device. Here, a predetermined space is formed in the inner portion of the rectangular vessel-shaped main body, support plates are formed on the bottom of the rectangular vessel-shaped main body and gas discharge and suction nozzles are located therein. In addition, a transparent see-through window is formed on the upper surface of the rectangular vessel-shaped main body, a detection electrode for controlling an inner atmosphere is formed at the center of the transparent see-through window, and one side surface of the rectangular vessel-shaped main body is opened to thus form a wafer inlet for introducing a wafer. The door opening and closing device includes: a forward and backward movement unit having cylinder loads, air cylinders, and plates, to make the door move forward and backward in order to open and close the wafer inlet of the main body of the VPD device; and an ascent and descent movement unit having a fixed plate, guide bars, a slide block, and a cylinder.

### CROSS-REFERENCE TO RELATED APPLICATION

This application claims the benefit of Korean Patent Application No. 10-2008-0031128, filed on Apr. 3, 2008, in the Korean Intellectual Property Office, the disclosure of which is incorporated herein by reference.

### BACKGROUND OF THE INVENTION

1. Field of the Invention

The present invention relates to a vapor phase decomposition (VPD) device for a semiconductor wafer pollutant measurement apparatus which is called an auto scanning system, and a door opening and closing device thereof, in which the semiconductor wafer pollutant measurement device scans components having sticked on the surface of a silicon wafer with a liquid sample, and thus measures a pollution level using the liquid sample.

2. Description of the Related Art

In general, as a semiconductor device is high-integrated, various kinds of pollutants that are produced in semiconductor manufacturing lines and semiconductor manufacturing processes are adsorbed on the surfaces of wafers. As a result, various kinds of pollutants that are adsorbed on the surfaces of wafers affect performance and yield of semiconductor devices.

Accordingly, an analysis of pollutants having sticked on the wafer surface has become important in manufacturing semiconductor devices. In the case of a conventional pollutant analysis method, a predetermined wafer is selected between the respective semiconductor manufacturing lines or the respective semiconductor manufacturing processes, and the surface of the selected wafer is scanned, to thus collect a sample of pollutants for analyzing the pollutants having sticked on the wafer surface, and analyze the collected pollutants sample using a destructive analysis method such as an atomic absorption spectroscopy and an inductively coupled plasma (ICP)-mass spectroscopy, and a nondestructive analysis method such as a total X-ray fluorescent analyzer.

That is, according to the conventional art, after the predetermined wafer has been selected between the respective semiconductor manufacturing lines or the respective semiconductor manufacturing processes, an oxide film that has been coated on the wafer surface should be removed before pollutants having sticked on the wafer surface are collected, in order to collect the pollutants accurately. This has been realized by a vapor phase decomposition (VPD) device.

The VPD device includes a process chamber in which a process proceeds, a loading plate which is positioned in the chamber and on which a wafer is loaded, and a container containing a hydrofluoric acid (HF) which decomposes an oxide film that is coated on the wafer surface. If a wafer is transferred on the loading plate which is installed in the process chamber, the wafer is safely placed in the process chamber for a given time. As a result, the oxide film that has been coated on the wafer surface is completely decomposed by vapor of the hydrofluoric acid (HF) which has been naturally evaporated from the hydrofluoric acid (HF) container.

Thereafter, a user takes out the wafer from the process chamber, and then drops a scanning solution on the wafer surface. The user scans the wafer surface directly manually, with the scanning solution, to thereby collect a sample of pollutants for pollution analysis of analyzing the pollutants having sticked on the wafer surface. Of course, the collected sample is analyzed to thereby measure a pollution level.

The Korean Patent Registration No. 10-0383264 entitled “Apparatus and method for collecting metallic impurity on a semiconductor wafer” corresponding to U.S. Patent Application Publication No. US 2002/0134406 A1 is already known as the semiconductor wafer pollutant measurement apparatus. The semiconductor wafer metallic impurity collecting apparatus generally includes a process chamber, a transfer unit, a loader unit, a vapor phase decomposition unit, a scanning unit, a dry unit, an unloader unit, and a center control unit that controls the semiconductor wafer metallic impurity collecting apparatus on the whole.

Here, the transfer unit, the loader unit, the vapor phase decomposition unit, the scanning unit, the dry unit, and the unloader unit among the components of the semiconductor wafer metallic impurity collecting apparatus are implemented in the process chamber. That is, the transfer unit, the loader unit, the vapor phase decomposition unit, the scanning unit, the dry unit, and the unloader unit are placed in a semi-circular form where the transfer unit is placed at the center of the semi-circular form, and the loader unit and the unloader unit are placed at the start and end portion of the semi-circular form, respectively. Here, the vapor phase decomposition unit, the scanning unit, and the dry unit are sequentially installed between the loader unit and the unloader unit.

The process chamber has an advantage of enhancing stability in operation since it is configured to have the transfer unit, the loader unit, the vapor phase decomposition unit, the scanning unit, the dry unit, and the unloader unit in a single closed space, but has a shortcoming that the units in the process chamber may be influenced when the wafer surface is decomposed by the atmosphere of the hydrofluoric acid (HF).

Various gases such as N₂ gas and air are injected into the process chamber to thereby keep cleanliness, in addition to decomposition of the hydrofluoric acid (HF), at the time of gathering a sample for measuring a pollution level of a wafer, and a predetermined gas pressure state should be maintained. Accordingly, in order to swiftly discharge gases which have been generated by decomposing the sample, injection and discharge of the gases should be accurately controlled. However, there is a problem that the conventional structure of the process chamber unnecessarily influences the transfer unit, the loader unit, the scanning unit, the dry unit, etc., in the process of decomposing the surface of the wafer gathered as a sample for measuring a pollution level of a wafer.

### SUMMARY OF THE INVENTION

To overcome inconveniences of the conventional art, it is an object of the present invention to provide an independent vapor phase decomposition (VPD) device, in which the VPD device is separated from the other devices in a semiconductor wafer pollutant measurement apparatus which is called an auto scanning system for measuring a pollution level of the wafer surface, to thus decompose the pollution level of the wafer surface using hydrofluoric acid (HF) and keep cleanness by N₂ gas.

It is another object of the present invention to provide a vapor phase decomposition (VPD) device, which can make a user see through the inside of the VPD device so that the inner air pressure and the injection state of gas can be easily controlled through the independent VPD device.

It is still another object of the present invention to provide a vapor phase decomposition (VPD) device for a semiconductor wafer pollutant measurement apparatus, in which an air pressure and a gas supply can be controlled at one space of the whole scanning system of the VPD device, to thereby increase a vapor phase decomposition effect of a wafer and to thus improve wafer yield.

It is yet another object of the present invention to provide a door opening and closing device for a vapor phase decomposition (VPD) device of a semiconductor wafer pollutant measurement apparatus, which heightens tightness of the VPD device and automates forward and backward operations and up and down operations of a door, to thereby make the door opened and closed quickly in response to a load of a wafer and a gas supply.

To accomplish the above object of the present invention, according to an aspect of the present invention, there is provided a vapor phase decomposition (VPD) device for use in a semiconductor wafer pollutant measurement apparatus, the VPD device comprising:

a rectangular vessel-shaped main body of the VPD device wherein a predetermined space is formed in the inner portion of the rectangular vessel-shaped main body, support plates are formed on the bottom of the rectangular vessel-shaped main body and gas discharge and suction nozzles are located therein, and wherein a transparent see-through window is formed on the upper surface of the rectangular vessel-shaped main body, a detection electrode for controlling an inner atmosphere is formed at the center of the transparent see-through window, and one side surface of the rectangular vessel-shaped main body is opened to thus form a wafer inlet for introducing a wafer; and

a door which covers in a sealed form or opens the wafer inlet of the VPD device.

According to another aspect of the present invention, there is provided a door opening and closing device for a vapor phase decomposition (VPD) device for use in a semiconductor wafer pollutant measurement apparatus, the door opening and closing device comprising:

a door which opens and closes a wafer inlet formed in a main body of the VPD device;

a forward and backward movement unit having air cylinders which are connected with cylinder loads, respectively, to make the door move forward and backward, and plates which support the air cylinders, to thereby open and close the wafer inlet of the main body of the VPD device; and

an ascent and descent movement unit having a slide block which is connected with the whole plates in order to make the forward and backward movement unit ascend and descend, a cylinder which makes the slide block move up and down, and guide bars which are located at either side of the cylinder, to guide the slide block to move up and down.

Preferably but not necessarily, the slide block is guided by an auxiliary guide bar at one side of the slide block.

### BRIEF DESCRIPTION OF THE DRAWINGS

The above and/or other aspects of the present invention will become apparent and more readily appreciated from the following description of the exemplary embodiments, taken in conjunction with the accompanying drawings in which:

FIG. 1 is a perspective view showing the inside of a conventional semiconductor wafer pollutant measurement apparatus;

FIG. 2A is a perspective view showing the whole external appearance of a semiconductor wafer pollutant measurement apparatus to which a vapor phase decomposition (VPD) device is applied;

FIG. 2B is a schematic plan view showing the inside of the semiconductor wafer pollutant measurement apparatus of FIG. 2A;

FIG. 3A is a perspective view showing a vapor phase decomposition (VPD) device for use in a semiconductor wafer pollutant measurement apparatus and a door opening and closing device thereof, according to the present invention;

FIG. 3B is a partially exploded perspective view showing the vapor phase decomposition (VPD) device according to the present invention;

FIG. 4A is a perspective view showing the door opening and closing device of the VPD device for the semiconductor wafer pollutant measurement apparatus, viewed from a certain direction, viewed from the bottom side, according to the present invention;

FIG. 4B is a perspective view showing the door opening and closing device of the VPD device for the semiconductor wafer pollutant measurement apparatus, viewed from another side, according to the present invention;

FIG. 4C is a side view showing the door opening and closing device of the VPD device for the semiconductor wafer pollutant measurement apparatus, according to the present invention;

FIGS. 5A and 5B are perspective views for explaining door opening and closing operations of the door opening and closing device for the semiconductor wafer pollutant measurement apparatus, according to the present invention, respectively;

FIG. 6A is a schematic diagram for explaining a state where a wafer is withdrawn using a robot arm in a main body of the VPD device according to the present invention;

FIG. 6B is a detailed perspective view showing a gas inlet and outlet unit in a main body of the VPD device for a semiconductor wafer pollutant measurement apparatus and the door opening and closing device thereof according to the present invention;

FIG. 7 is a schematic diagram for explaining gas control when the VPD device for a semiconductor wafer pollutant measurement apparatus and the door opening and closing device thereof according to the present invention are actually applied; and

FIG. 8 is a flowchart view showing operations of opening and closing a door when a wafer is input to the main body of the VPD device for a semiconductor wafer pollutant measurement apparatus and the door opening and closing device thereof according to the present invention.

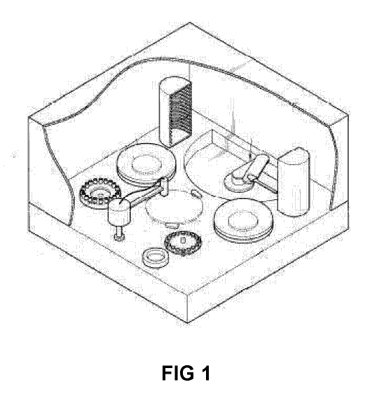

### DETAILED DESCRIPTION OF THE INVENTION

Hereinbelow, a vapor phase decomposition (VPD) device for a semiconductor wafer pollutant measurement apparatus which is called an auto scanning system, and a door opening and closing device thereof, according to the present invention will be described with reference to the accompanying drawings. Like reference numerals are assigned for like elements in the drawings.

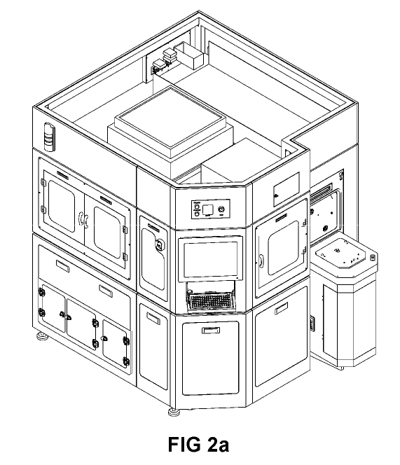

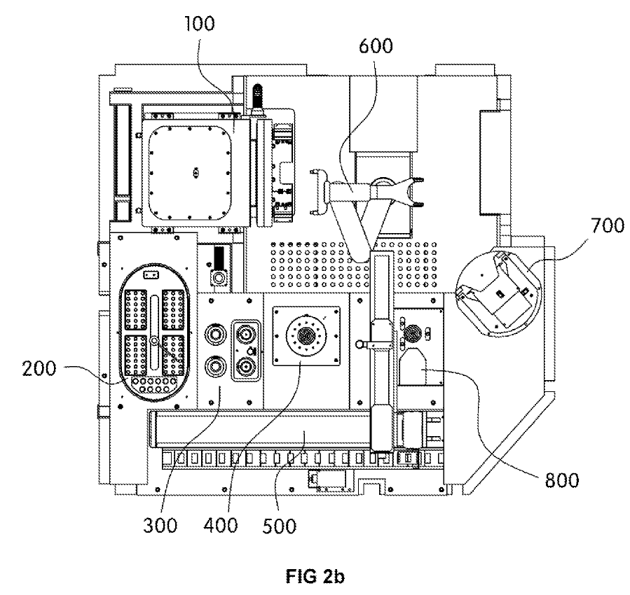

FIG. 2A is a perspective view showing the whole external appearance of a semiconductor wafer pollutant measurement apparatus to which a vapor phase decomposition (VPD) device is applied, and FIG. 2B is a schematic plan view showing the inside of the semiconductor wafer pollutant measurement apparatus of FIG. 2A.

As shown in FIGS. 2A and 2B, a vapor phase decomposition (VPD) device for a semiconductor wafer pollutant measurement apparatus according to the present invention, includes: an opener 700 which is located at an entrance portion in a space which is isolated from the outside portion, and opens a wafer cassette according to size of a wafer such as 300 mm, 200 mm, and 150 mm, for example; a robot arm 600 that picks up a wafer in a cassette that is safely loaded into the opener 700 and transfers the picked wafer; a VPD device 100 according to the present invention which primarily decomposes an oxide film formed on the surface of the wafer that is transferred by the robot arm 600; a scan stage 400 which supports the wafer whose oxide film has been decomposed and rotates the wafer at a state where the wafer has been supported; and a scanning unit 500 which moves along three axes of x, y and z, and inhales a reagent from an inhalation and washing unit 300 and then moves the scan stage 400 to thus perform a scanning operation on the wafer put on the scan stage 400, in order to make the wafer put on the scan stage 400 contact a solution, and to thereafter discharge the scanned solution into a bottle of an analyser unit 200. After having discharged, the inhalation and discharge nozzle of the scanning unit 500 is washed in the inhalation and washing unit 300 to then inhale the reagent from the inhalation and washing unit 300 and to repeat a scanning operation.

Description of scanning will be omitted. Here, if the scanning unit 500 reaches a predetermined position of a wafer at a state where a predetermined amount of solution is inhaled into the nozzle, it rotates the wafer and discharges part of the solution to contact the surface of the wafer. In this case, if the scanning unit 500 moves along most surfaces of the wafer in a straight line, and makes the wafer rotate, it makes most surfaces of the wafer contact the solution so that pollutants can be collected. The solution of the collected pollutants are transferred to the analyzer unit 200 at a state where the nozzle is in an inhalation position to then be discharged into an empty bottle of the analyzer unit 200.

Thereafter, the transfer unit robot arm 600 transfers the wafers whose tests are finished to an aligner unit 800, to thereby make the aligner unit 800 recognize identification (ID) of each wafer and align the wafers, to then remove the aligned wafers into a cassette.

If the solution having included the pollutants is discharged to the bottle of the analyzer unit 200, the whole operation of the VPD device of this invention ends. Then, a user measures a pollution level in every bottle through a semiconductor wafer pollutant measurement apparatus.

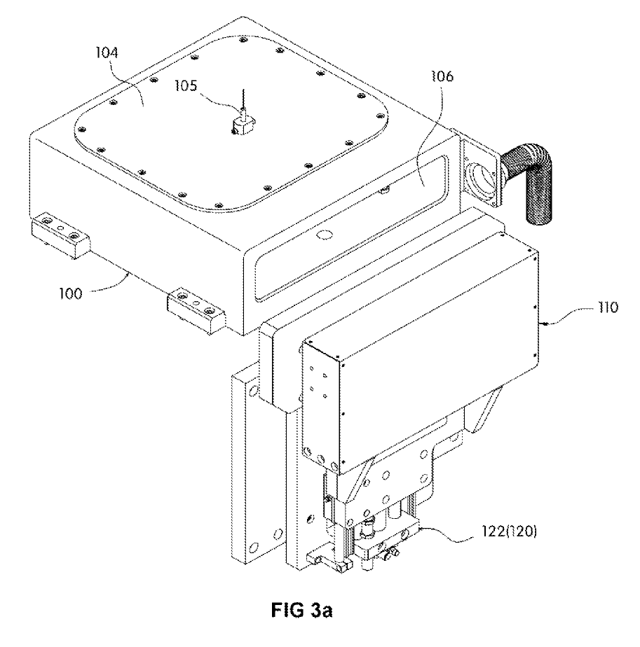

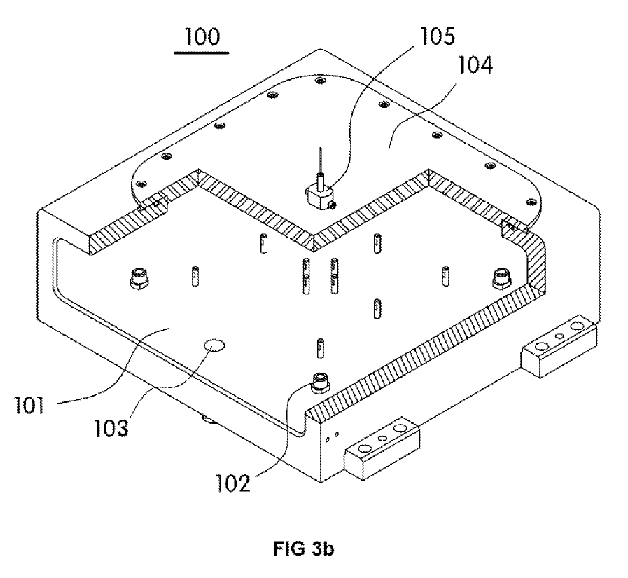

FIG. 3A is a perspective view showing a vapor phase decomposition (VPD) device for use in a semiconductor wafer pollutant measurement apparatus and a door opening and closing device thereof, according to the present invention, and FIG. 3B is a partially exploded perspective view showing the vapor phase decomposition (VPD) device for use in a semiconductor wafer pollutant measurement apparatus, according to the present invention.

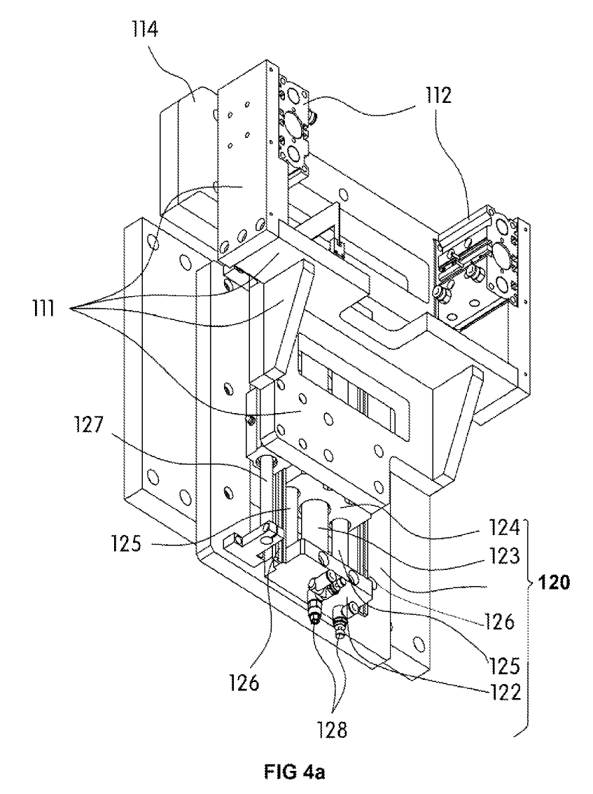

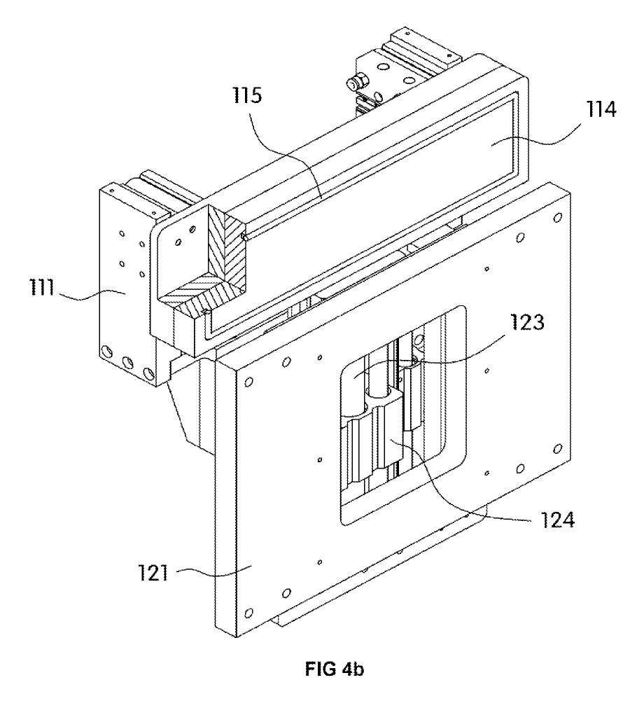

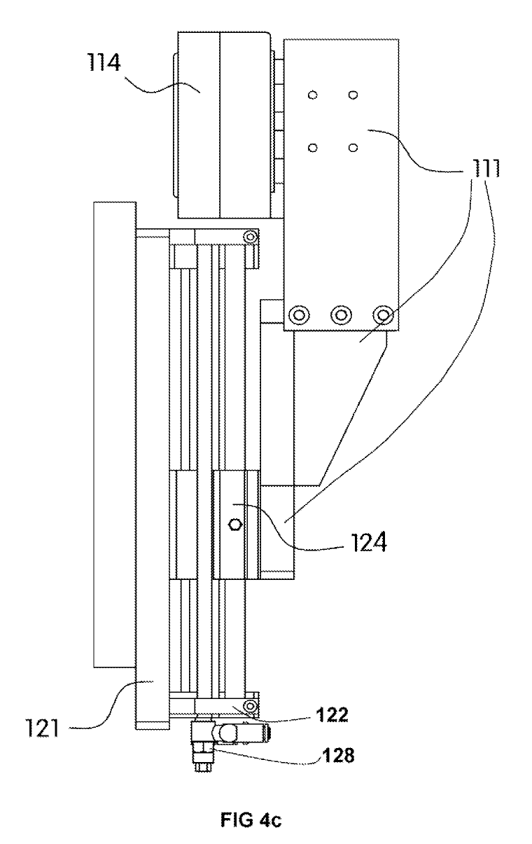

As shown in FIGS. 3A and 3B, a VPD device for a semiconductor wafer pollutant measurement apparatus according to the present invention, includes: a main body 100 and a door 114 (of FIG. 4A or 4B) having a door opening and closing device 110 and 120.

A space 101 is formed in the inside of the main body 100. A plurality of nozzles 102 through which a fluorine gas and N₂ gas are injected and an outlet 103 through which the decomposed gases are discharged are formed in the space 101 of the main body 100. A transparent see-through window 104 is formed on the upper surface of the main body 100, and a detection electrode 105 for detecting a situation after having decomposed the gases in the main body 100 is formed at the center of the transparent see-through window 104.

A door 114 (of FIG. 4A or 4B) is a generally rotatable door having a tightness function which closes an inlet 106 of the main body 100. The VPD device 100 according to the present invention is implemented into a state where a door 114 has been installed in an independently operable door opening and closing device. As will be described later, it is preferable that the door in the present invention is configured to operate by the door opening and closing device, but may be implemented into a rotary type door which will be omitted since it is apparent to one skilled in the art.

The door opening and closing device includes: a forward and backward movement unit 110 and an ascent and descent movement unit 120, and moves forward and backward, and up and down, from the front surface of the inlet 106 of the main body 100 of the VPD device, to thus open and close the inlet 106.

FIG. 4A is a perspective view showing the door opening and closing device of the VPD device for the semiconductor wafer pollutant measurement apparatus, viewed from a certain direction, viewed from the bottom side, according to the present invention. FIG. 4B is a perspective view showing the door opening and closing device of the VPD device for the semiconductor wafer pollutant measurement apparatus, viewed from another side, according to the present invention. FIG. 4C is a side view showing the door opening and closing device of the VPD device for the semiconductor wafer pollutant measurement apparatus, according to the present invention.

As shown in FIGS. 4A to 4C, the door opening and closing device includes: the forward and backward movement unit 110 and the ascent and descent movement unit 120. Firstly, the forward and backward movement unit 110 will be described below.

The forward and backward movement unit 110 includes: air cylinders 112 which are connected with cylinder loads 113, respectively, to make a door 114 move forward and backward, and plates 111 which support the air cylinders 112, to thereby open and close the wafer inlet 106 of the main body 100 of the VPD device. Here, the door 114 is of a flat plate style to open and close the inlet 106 of the VPD device.

As shown, the plates 111 include: a fork style upper portion both sides of which are connected with the air cylinders 112; and a connection portion which connects the fork style upper portion with the ascent and descent movement unit 120. Since the plates 111 are integrally connected with a fixed plate 121 in which the ascent and descent movement unit 120 has been mounted, the whole parts of the forward and backward movement unit 110 and the ascent and descent movement unit 120 both of which are integrated with the door 114 have a two-axis movement mechanism of up-and-down movement and forward and backward movement.

The ascent and descent movement unit 120 includes: the fixed plate 121 which is connected with the plates 111 in order to make the forward and backward movement unit 110 ascend and descend; guide bars 125 which are installed at both sides of the fixed plate 121 in a ball bushing structure; sensor frames 126 having sensors which are provided at both sides of the sensor frames 126 to sense ascent and descent heights at the outer side of the guide bars 125, respectively; an auxiliary guide bar 127 which stably guides ascending and descending of the whole plates 111 at one side of the auxiliary guide bar 127; and a cylinder 123 which makes the slide block 124 move up and down at the center of the slide block 124.

The cylinder 123 which is applied in the present invention is of a cylindrical vessel structure and is communicated with a cylinder block 122 at the upper and lower ends of the cylinder 123, in a communicating structure between the cylinder 123 and the cylinder block 122. Connection units 128 for injecting and discharging air are installed at the lower side of the cylinder 123. In particular, pieces of magnets (not shown) which move up and down by an air pressure are built in the inside of the cylinder 123, and the slide block 124 maintains adsorption by magnetic forces of the magnet pieces. The slide block 124 is connected with the lower portion of the plates 111 of the forward and backward movement unit 110.

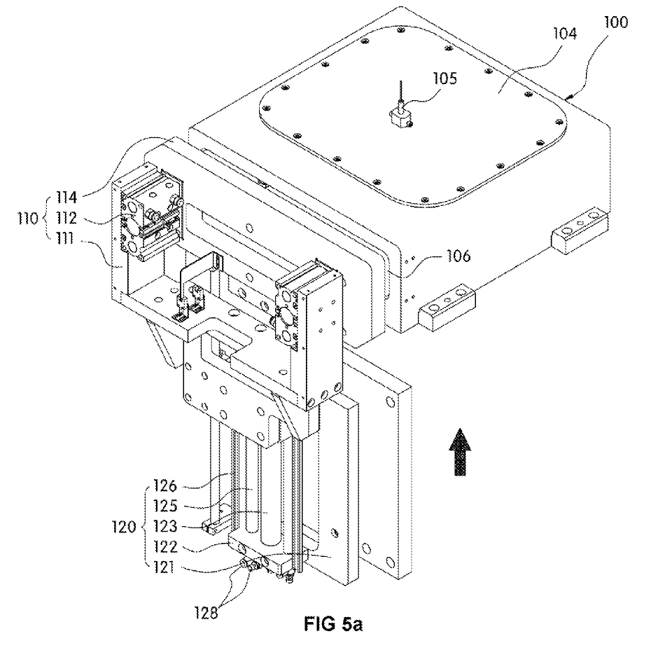

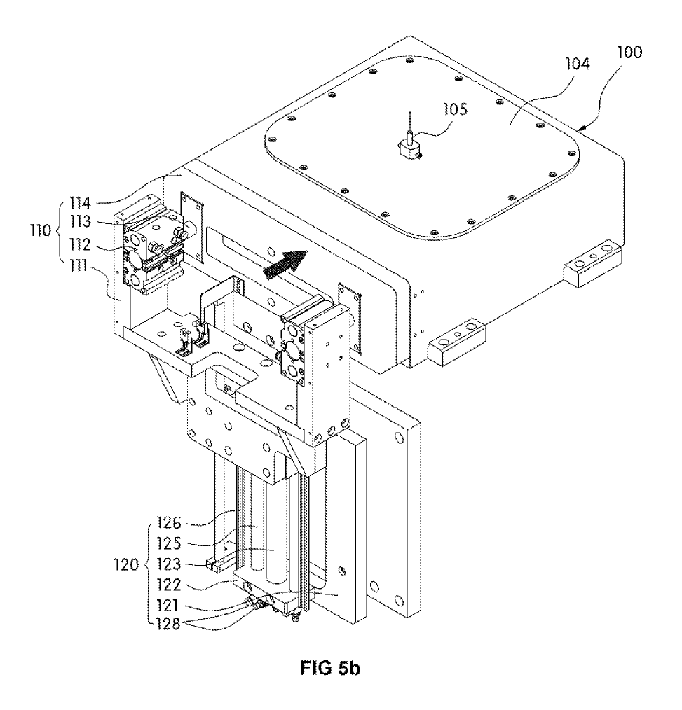

FIGS. 5A and 5B are perspective views for explaining door opening and closing operations of the door opening and closing device for the semiconductor wafer pollutant measurement apparatus, according to the present invention, respectively.

As shown in FIGS. 5A and 5B, in operation, the door 114 of the main body 100 is made to move up and down and forward and backward by the air cylinders 112, to thus open and close the door 114 of the main body 100 of the VPD device.

After a wafer has been put in the main body 100 of the VPD device, air of a predetermined pressure is supplied via the connection units 128 of the cylinder block 122 in order to make the slide block 124 integrated with the plates 111 of the forward and backward movement unit 110 integrated with the door 114. The magnet pieces positioned in the cylinder 123 move up by injection of air. Accordingly, the slide block 124 whose adsorption is maintained by the magnetic forces of the magnet pieces moves. When the slide block 124 moves, the whole parts of the plates 111 of the forward and backward movement unit 110 integrated with the slide block 124 move up to a predetermined height.

If the door 114 reaches at a positive position by the ascending operation of the ascent and descent movement unit 120, the ascending operation stops according to a sensed signal, and then the cylinders 112 of the forward and backward movement unit 110 operate. The door 114 which is connected with the loads 113 closely contacts the inlet 106 of the main body 106 of the VPD device by the forward movement of the cylinders 112, to thereby isolate the inner and outer portions of the main body 100 to thus complete a closing operation.

Thereafter, when etching of the wafer surface has been completed, the cylinders 112 moves backward in order to open the door 114. In sequence, the whole plates 111 descend by operation of the cylinders 123 of the ascent and descent movement unit 120, and are restored into original positions, respectively.

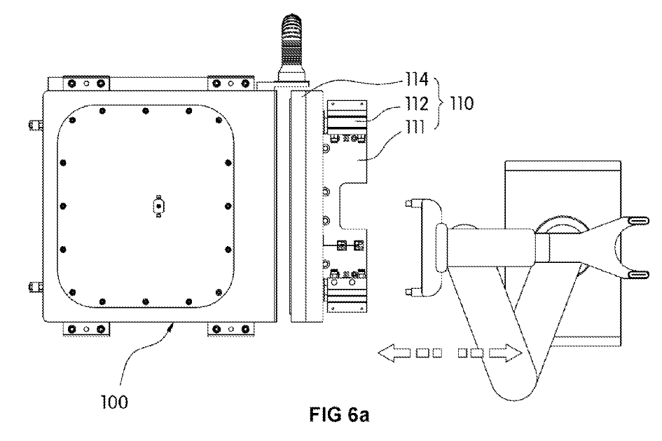

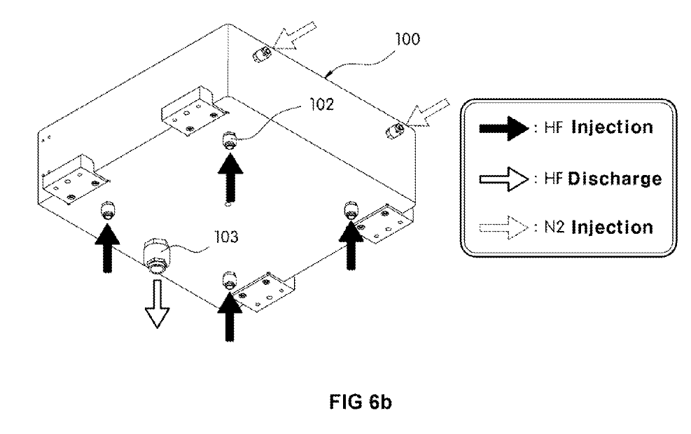

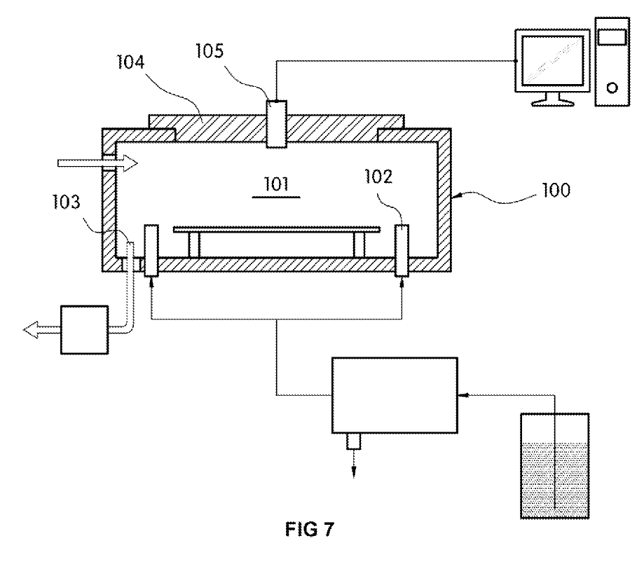

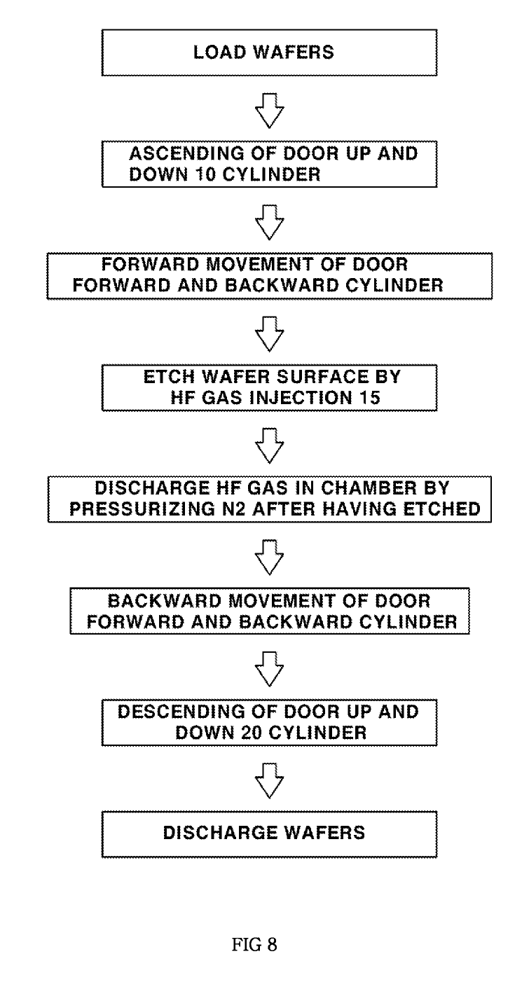

FIG. 6A is a schematic diagram for explaining a state where a wafer is withdrawn using a robot arm in a main body of the VPD device according to the present invention. FIG. 6B is a detailed perspective view showing a gas inlet and outlet unit in a main body of the VPD device for a semiconductor wafer pollutant measurement apparatus and the door opening and closing device thereof according to the present invention. FIG. 7 is a schematic diagram for explaining gas control when the VPD device for a semiconductor wafer pollutant measurement apparatus and the door opening and closing device thereof according to the present invention are actually applied, and FIG. 8 is a flowchart view showing operations of opening and closing a door when a wafer is input to the main body of the VPD device for a semiconductor wafer pollutant measurement apparatus and the door opening and closing device thereof according to the present invention.

As illustrated, a HF gas is injected via nozzles 102 in the main body 100 of the VPD device according to the present invention, and thus the wafer surface which is positioned in the inner space of the main body 100 is etched. After having etched, a N₂ gas is injected into the inside of the main body 100 with a predetermined pressure, and the HF gas existing in the main body 100 of the VPD device is discharged via an outlet 103. After having discharged the HF gas, the door 114 moves backward by the cylinders 112 of the forward and backward movement unit 110, to thus make the inlet 106 opened. The backwardly moved door 114 moves down by the cylinders 122 of the ascent and descent movement unit 120. Thereafter, the wafers placed in the inside of the main body 100 are taken out by a wafer transfer robot.

Then, ingredients of silicone (Si) wafer surface are scanned by a liquid sample and a pollution level of the liquid sample is measured in a semiconductor wafer pollutant measurement apparatus. That is, since the wafer surface is exposed to the HF gas in the sealed main body 100, metal components are oxidized, and the etched wafer surface is scanned, to thus collect a liquid sample. The collected sample is transferred to a clean sample bottle by a pipette of a predetermined transfer unit. The metal components of the collected sample solution is analyzed by an ICP-MS (Inductively Coupled Plasma-Mass Spectrometry).

To analyze a small quantity of metal ingredients on the silicone (Si) wafer, ICP-MS is used. Droplets extracted in the scanning process are extracted to grasp a pollution level of the surface of the droplets, and wafer sample preparation and analysis are executed within 30 minutes, and enables real-time monitoring when manufacturing wafers.

The present invention is not limited to the above-described embodiments. It is apparent to one who has an ordinary skill in the art that there may be many modifications and variations within the same technical spirit of the invention.

As described above, the present invention provides an independent vapor phase decomposition (VPD) device, in which the VPD device is separated from the other devices in a semiconductor wafer pollutant measurement apparatus which is called an auto scanning system for measuring a pollution level of the wafer surface, to thus decompose the pollution level of the wafer surface using hydrofluoric acid (HF) and keep cleanness by N₂ gas.

The present invention provides a vapor phase decomposition (VPD) device, which can make a user see through the inside of the VPD device so that the inner air pressure and the injection state of gas can be easily controlled through the independent VPD device.

The present invention provides a vapor phase decomposition (VPD) device for a semiconductor wafer pollutant measurement apparatus, in which an air pressure and a gas supply can be controlled at one space of the whole scanning system of the VPD device, to thereby increase a vapor phase decomposition effect of a wafer and to thus improve wafer yield.

The present invention provides a door opening and closing device for a vapor phase decomposition (VPD) device of a semiconductor wafer pollutant measurement apparatus, which heightens tightness of the VPD device and automates forward and backward operations and up and down operations of a door, to thereby make the door opened and closed quickly in response to a load of a wafer and a gas supply.

What is claimed is:

1. A vapor phase decomposition (VPD) device for use in a semiconductor wafer pollutant measurement apparatus, the VPD device comprising:
a rectangular vessel-shaped main body of the VPD device wherein a predetermined space is formed in the inner portion of the rectangular vessel-shaped main body, support plates are formed on the bottom of the rectangular vessel-shaped main body and gas discharge and suction nozzles are located therein, and wherein a transparent see-through window is formed on the upper surface of the rectangular vessel-shaped main body, a detection electrode for controlling an inner atmosphere is formed at the center of the transparent see-through window, and one side surface of the rectangular vessel-shaped main body is opened to thus form a wafer inlet for introducing a wafer; and
a door which covers in a sealed form or opens the wafer inlet of the VPD device.

2. A door opening and closing device for a vapor phase decomposition (VPD) device for use in a semiconductor wafer pollutant measurement apparatus, the door opening and closing device comprising:
a door which opens and closes a wafer inlet formed in a main body of the VPD device;
a forward and backward movement unit having air cylinders which are connected with cylinder loads, respectively, to make the door move forward and backward, and plates which support the air cylinders, to thereby open and close the wafer inlet of the main body of the VPD device; and
an ascent and descent movement unit having a slide block which is connected with the whole plates in order to make the forward and backward movement unit ascend and descend, a cylinder which makes the slide block move up and down, and guide bars which are located at either side of the cylinder, to guide the slide block to move up and down.

3. The door opening and closing device according to claim 1, wherein the slide block is guided by an auxiliary guide bar at one side of the slide block.
